(19) United States
(12) Patent Application Publication
(10) Pub. No.: US 2019/0059809 A1
(43) Pub. Date: Feb. 28, 2019

(54) METHOD AND APPARATUS FOR DETERMINING A PAIN THRESHOLD OF A SUBJECT

(71) Applicant: KONINKLIJKE PHILIPS N.V., EINDHOVEN (NL)

(72) Inventors: Dirkes MARCEL CORNELIS, The Hague (NL); Jens MUEHLSTEFF, Aachen (DE); Johannes WEDA, Nijmegen (NL)

(73) Assignee: KONINKLIJKE PHILIPS N.V., EINDHOVEN (NL)

(21) Appl. No.: 15/763,244

(22) PCT Filed: Sep. 27, 2016
(86) PCT No.: PCT/EP2016/072987

§ 371 Date: Mar. 26, 2018

(30) Foreign Application Priority Data
Sep. 29, 2015 (EP) 15187358.5

(51) Int. Cl. A61B 5/00; A61B 5/0205; A61B 5/107

(52) U.S. Cl. CPC A61B 5/4824; A61B 5/0205; A61B 5/024; A61B 5/7285; A61B 5/1071; A61B 5/7278

(57) ABSTRACT

There is provided an apparatus for determining a pain threshold of a subject, the apparatus comprising a processing unit adapted to monitor the subject to identify a suitable time for determining the pain threshold of the subject; control a first electrode to deliver an electric signal to a sural nerve of the subject at an identified suitable time; receive a first output signal from a first sensor that measures a reaction of a muscle of the subject to the electric signal; and determine the pain threshold of the subject from the received first output signal.

### TECHNICAL FIELD OF THE INVENTION

The invention relates to a method and apparatus for determining a pain threshold of a subject, and in particular relates to a method and apparatus for determining a pain threshold of a subject in which an electric signal is delivered to a sural nerve of the subject.

### BACKGROUND TO THE INVENTION

Pain monitoring is required for post-operative patients in order to provide adequate analgesia and prevent dangerous side-effects such as opioid-induced respiratory depression. However, the pain level of a subject is often subjective. When medication (e.g. an analgesic) is required, an objective measurement of pain would help a physician in providing an adequate prescription for a subject. It has been found that an objective measurement of pain can be made using mild stimulation of the sural nerve and observation of the subsequent muscular response (specifically of the vastus lateralis).

The sural nerve is an entirely sensory nerve (as opposed to most other nerves in the body that combine sensory and motor function). Stimulation of the sural nerve therefore does not produce a direct movement of a part of the body e.g. the foot. However, at a certain stimulation threshold, this nerve relays the signal in the spine to produce a reflex flexion of the quadriceps muscle in the thigh. It has been demonstrated that the threshold at which this reflex is initiated is indicative of the pain threshold of a subject, and that it responds to analgesics in a proportional manner.

WO 2011/054959 describes an existing method for quantifying the reflex response to a stimulation signal using electromyography (EMG) of the upper thigh (specifically the vastus lateralis).

### SUMMARY OF THE INVENTION

It can be desirable for a measurement of the pain threshold of a subject to be made frequently or at regular intervals, particularly for a post-operative subject in a healthcare environment. However, the technique in WO 2011/054959 requires a subject to be seated or lying down and relaxed in order to minimize interference from voluntary motor activity evoked EMG signals. Thus, if a subject is mobile, which is important for post-operative rehabilitation, it may be difficult to successfully use sural nerve stimulation for pain threshold monitoring, more specifically it may not be possible to measure the muscle response, and/or the measurements may give misleading or inaccurate measurements. A similar problem exists when measuring the pain threshold of a subject that is in their home environment, since they will also frequently be mobile.

Therefore there is a need for an improved method and apparatus for determining a pain threshold of a subject.

According to a first aspect, there is provided an apparatus for determining a pain threshold of a subject, the apparatus comprising a processing unit adapted to monitor the subject to identify a suitable time for determining the pain threshold of the subject; control a first electrode to deliver an electric signal to a sural nerve of the subject at an identified suitable time; receive a first output signal from a first sensor that measures a reaction of a muscle of the subject to the electric signal; and determine the pain threshold of the subject from the received first output signal.

In some embodiments the first sensor is an accelerometer, a stretch sensor, a strain-gauge sensor or a second electrode.

In some embodiments the processing unit is adapted to process an output signal from the first sensor to identify a suitable time for determining the pain threshold of the subject.

In some embodiments the processing unit is further adapted to receive a second output signal from a second sensor, and to process the second output signal to identify the suitable time for determining the pain threshold of the subject. The second sensor can be an accelerometer.

In some embodiments the processing unit is adapted to monitor a heart rate of the subject to identify the suitable time for determining the pain threshold of the subject. In some embodiments the processing unit is adapted to compare the heart rate of the subject to a threshold, and to identify the suitable time for determining the pain threshold of the subject as a time at which the heart rate of the subject is below the threshold.

In some embodiments the processing unit is adapted to monitor movements of the muscle of the subject to identify the suitable time for determining the pain threshold of the subject.

In some embodiments the processing unit is adapted to identify the suitable time for determining the pain threshold of the subject as a time in which an activity level of the muscle of the subject is below a threshold.

In some embodiments the processing unit is adapted to monitor a posture of the leg of the subject to identify the suitable time for determining the pain threshold of the subject. In some embodiments the processing unit is adapted to identify the suitable time for determining the pain threshold of the subject as the time in which the knee of the subject is bending at an angle that is within a predetermined range of angles.

In some embodiments, the apparatus further comprises the first electrode and/or the first sensor.

According to a second aspect, there is provided a method of determining a pain threshold of a subject, the method comprising the steps of monitoring the subject to identify a suitable time for determining the pain threshold of the subject; controlling a first electrode to deliver an electric signal to a sural nerve of the subject at an identified suitable time; receiving a first output signal from a first sensor that measures a reaction of a muscle of the subject to the electric signal; and determining the pain threshold of the subject from the received first output signal.

In some embodiments the first sensor is an accelerometer, a stretch sensor, a strain-gauge sensor or a second electrode.

In some embodiments the step of monitoring the subject to identify a suitable time for determining the pain threshold of the subject comprises processing an output signal from the first sensor to identify the suitable time.

In some embodiments the method further comprises the step of receiving a second output signal from a second sensor, and the step of monitoring the subject to identify a suitable time for determining the pain threshold of the subject comprises processing the second output signal to identify the suitable time. The second sensor can be an accelerometer.

In some embodiments the step of monitoring the subject to identify a suitable time for determining the pain threshold of the subject comprises monitoring a heart rate of the subject to identify the suitable time. In some embodiments the step of monitoring the heart rate comprises comparing the heart rate of the subject to a threshold, and identifying the suitable time for determining the pain threshold of the subject as a time at which the heart rate of the subject is below the threshold.

In some embodiments the step of monitoring the subject to identify a suitable time for determining the pain threshold of the subject comprises monitoring movements of the muscle of the subject to identify the suitable time for determining the pain threshold of the subject.

In some embodiments the step of monitoring the subject to identify a suitable time for determining the pain threshold of the subject comprises identifying the suitable time for determining the pain threshold of the subject as a time in which an activity level of the muscle of the subject is below a threshold.

In some embodiments the step of monitoring the subject to identify a suitable time for determining the pain threshold of the subject comprises monitoring a posture of the leg of the subject to identify the suitable time for determining the pain threshold of the subject. In some embodiments the step of monitoring the subject to identify a suitable time for determining the pain threshold of the subject comprises identifying the suitable time as a time in which the knee of the subject is bending at an angle that is within a predetermined range of angles.

According to a third aspect, there is provided a computer program product comprising a computer readable medium having computer readable code embodied therein, the computer readable code being configured such that, on execution by a suitable computer or processor, the computer or processor is caused to perform any of the methods described above.

According to a fourth aspect there is provided an apparatus for determining a pain threshold of a subject, the apparatus comprising a processing unit adapted to: monitor the subject to identify a suitable time for determining the pain threshold of the subject; control a first electrode to deliver an electric signal to a sural nerve of the subject at an identified suitable time; receive a first output signal from a first sensor that measures a reaction of a muscle of the subject to the electric signal; and determine the pain threshold of the subject from the received first output signal; wherein the processing unit is adapted to, based on a sensor output, monitor a heart rate of the subject, monitor movements of the muscle of the subject, and/or monitor a posture of the leg of the subject, in order to identify the suitable time for determining the pain threshold of the subject.

According to a fifth aspect there is provided a method of determining a pain threshold of a subject, the method comprising: monitoring the subject to identify a suitable time for determining the pain threshold of the subject; controlling a first electrode to deliver an electric signal to a sural nerve of the subject at an identified suitable time; receiving a first output signal from a first sensor that measures a reaction of a muscle of the subject to the electric signal; and determining the pain threshold of the subject from the received first output signal; wherein monitoring the subject to identify the suitable time for determining the pain threshold of the subject comprises monitoring a heart rate of the subject, monitoring movements of the muscle of the subject, and/or monitoring a posture of the leg of the subject.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 shows an apparatus according to an aspect of the invention;

FIG. 2 shows the apparatus of FIG. 1 being worn by a subject;

FIG. 3 is a flow chart illustrating a method of determining a pain threshold of a subject according to an aspect;

FIG. 4 is a diagram illustrating the processing performed by the processing unit according to an exemplary embodiment; and

FIG. 5 is a series of graphs illustrating measurements obtained from a stretch sensor attached to the thigh of a subject.

### DETAILED DESCRIPTION OF THE PREFERRED EMBODIMENTS

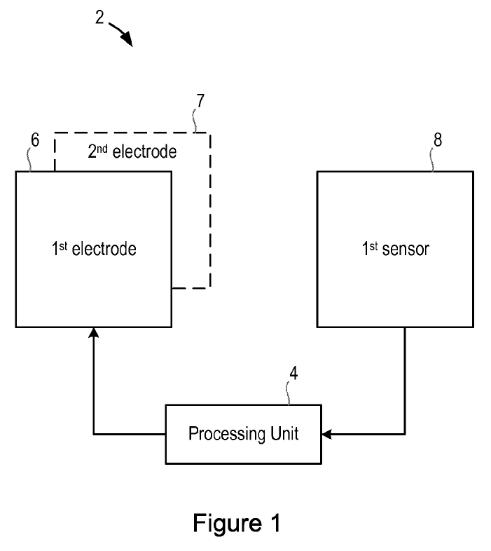

FIG. 1 is a block diagram of an apparatus 2 according to an aspect of the invention. The apparatus 2 comprises a processing unit 4 that controls the operation of the apparatus 2 and that can implement the pain threshold measurement method. The processing unit 4 is configured or adapted to receive a signal from a sensor that is monitoring the part of the body of the subject and to process the sensor signal to determine the pain threshold of the subject. The processing unit 4 may receive a signal directly from the sensor (e.g. as the sensor monitors the part of the body of the subject), or retrieve previously-obtained sensor measurements from a memory unit (not shown in FIG. 1). The processing unit 4 can comprise one or more processors, control units, multi-core processors or processing modules that are configured or programmed to control the apparatus 2 to determine the pain threshold of a subject as described below.

The processing unit 4 may also be configured or adapted to cause or control an electrode to apply an electric signal to a part of the body of a subject to cause a muscle in the subject's body to contract or twitch (reflex). The processing unit 4 may be configured or adapted to cause or control an electrode to apply electric signals of different magnitudes to the part of the body of the subject. In particular, the processing unit 4 may be configured or adapted to provide an electric signal of varying current to an electrode. The current may be of the order of a few milliAmps, and thus the current may be varied between 1 mA and 50 mA, for example.

In the embodiment illustrated in FIG. 1, the apparatus 2 further comprises one or more electrodes 6 (e.g. a first electrode 6 and a second electrode 7) that arc connected to the processing unit 4 and a sensor 8 that is connected to the processing unit 4 for measuring the reaction of a muscle to an electric signal delivered by the one or more electrodes 6, 7. The one or more electrodes 6 and/or the sensor 8 may be connected to the processing unit 4 via one or more wires or leads. In alternative embodiments, the electrode(s) and/or the sensor can be separate from the apparatus 2 and can be selectively attached or connected (via wires or wirelessly) to the apparatus 2/processing unit 4 as required.

In some embodiments, the processing unit 4 may be part of a smart phone or other general purpose computing device that can be connected to electrode(s) 6, but in other embodiments the apparatus 2 can be an apparatus that is dedicated to the purpose of measuring the pain threshold of a subject. In embodiments where the processing unit 4 is part of a smart phone or other general purpose computing device, the sensor 8 could be a sensor that is integrated into the smart phone, or a sensor that is separate to the smart phone and that can provide sensor signals/measurements to the smart phone/computing device for processing and analysis (for example via a wired or wireless connection).

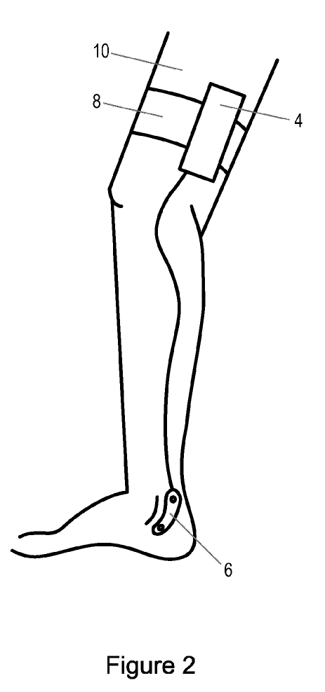

The one or more electrodes 6 are to be used to deliver an electric signal to a sural nerve of a subject. As noted above, the sural nerve is an entirely sensory nerve that, at a certain stimulation threshold, relays a signal in the spine to produce a reflex flexion of the quadriceps muscle in the thigh. The sural nerve can be stimulated by electrode(s) 6 placed at or near one of the ankles of a subject 10, as shown in FIG. 2. In particular, the electrode(s) 6 should be placed on the dorsal side of the lateral malleolus, directly above the path of the sural nerve.

The electrode(s) 6 can be any suitable type of electrode for delivering an electric signal to a body of a subject, and those skilled in the art will be aware of various types of electrodes that can be used in the apparatus 2 according to the invention. In addition, the electrode(s) 6 can be attached to the body of the subject by any suitable means, for example via an adhesive, strap, etc.

In some embodiments the processing unit 4 comprises suitable circuitry for generating the electric signal to be delivered to the subject 10 by the electrode(s) 6, whereas in other embodiments there may be additional processing or circuitry components that generate the electric signal to be delivered to the subject 10 by the electrode(s) 6 in response to a control signal from the processing unit 4.

As noted above, the sensor 8 is used to measure the reaction of a muscle to an electric signal delivered by the one or more electrodes 6. In particular, the sensor 8 is used to measure the reaction of a muscle in the leg of a subject 10, and in particular the reaction of the vastus lateralis to an electric signal applied to the sural nerve of that leg. Preferably the sensor 8 is located on or near the muscle to be monitored, e.g. at the level of the vastus lateralis, and thus the sensor 8 can be attached to or otherwise in contact with the thigh of the subject 10 (on the same leg that the electrode(s) 6 are attached to), as shown in FIG. 2.

The sensor 8 can be of any suitable type for enabling a muscle response (e.g. a twitch) to be measured. For example, the sensor 8 can be an accelerometer that is to be placed in contact with the thigh of the subject 10 to measure accelerations experienced at that location, and the processing unit 4 can process the acceleration signal to identify accelerations caused by the twitching of the muscle. The accelerometer 8 can be an accelerometer that measures accelerations in three dimensions.

In an alternative embodiment, the sensor 8 can be a stretch sensor that is placed around the leg (a stretch sensor is shown in FIG. 2) and that measures changes in the volume of the leg under the sensor caused by movements (e.g. contractions) of muscle(s), and the processing unit 4 can process the signal from the stretch sensor to identify stretches caused by the twitching of the muscle. Those skilled in the art will appreciate that a stretch sensor can comprise a piece of material whose electrical properties change when it is stretched, compressed or bent. As an alternative to a stretch sensor, a strain gauge sensor could be used to identify stretches caused by the twitching of the muscle.

In yet another embodiment, the twitches of the muscle can be measured using electromyography (EMG), and thus the sensor 8 can comprise one or more electrodes (different to the electrode(s) 6 that are used to stimulate the sural nerve) that are placed on the skin of the leg of the subject 10 to measure the electrical activity produced by the muscles at that location. The processing unit 4 can process the signals from the electrodes to identify twitches of the muscle. Those skilled in the art are aware of techniques for processing EMG signals to identify muscle twitches e.g. as described in WO 2011/054959, and therefore further details are not provided herein.

Although EMG can be used to measure the twitches of the muscle according to the invention, EMG suffers from a number of drawbacks compared to the use of an accelerometer or a stretch sensor. In particular, EMG signals are sensitive to noise, especially to 50/60 Hz noise from AC (alternating current) power lines, lights, relays, and transformers; the electrodes need to be applied to the skin, and the skin needs to be prepared for optimal sensor contact (e.g. cleaning, scrubbing, applying conductive paste or gel, etc.); and EMG signals are sensitive to the exact positioning of the electrodes, which includes the size and distance between the electrodes. In view of these drawbacks, and other reasons discussed below, the use of an accelerometer or stretch sensor to measure the twitches of the muscle according to the invention is preferred, particularly for apparatus 2 that is to be used by a subject at home, since an accelerometer or stretch sensor can be more easily positioned correctly on the subject 10, and direct contact with the skin of the subject 10 is not required.

Aside from an accelerometer, stretch sensor, strain gauge and EMG, those skilled in the art will be aware of other types of sensors that can be used to measure or detect a twitch in a muscle, and the apparatus 2 described herein is not intended to be limited to any particular type of sensor 8.

It will be appreciated that in some embodiments the apparatus 2 can make use of multiple sensors 8 (of the same or different types) to detect muscle twitches to improve the reliability of the twitch detection.

To improve the ease with which the subject 10 can correctly position the electrode(s) 6 and/or sensor 8 on their body, the electrode(s) 6 and/or sensor 8 can be integrated into one or more wearable items, e.g. a sock or plaster in the case of the electrode(s) 6, a piece of cloth or band in the case of the sensor 8.

Although not shown in FIG. 1, the apparatus 2 can further comprise a memory module for storing program code that can be executed by the processing unit 4 to perform the method described herein. The memory module can also be used to store signal and measurements made or obtained by the apparatus 2 during operation.

As noted in more detail below, to improve the reliability of measurements of the pain threshold of a subject, the invention avoids making measurements when the subject is not resting and/or in an appropriate posture for the measurement. In particular, the invention provides that a suitable time at which a measurement of the pain threshold could be made is identified. In certain embodiments, a suitable time is a time where the subject 10 is at rest (e.g. as indicated by a low heart rate and/or low levels of physical activity), and/or when the leg of the subject 10 is in an appropriate posture (e.g. in which the vastus lateralis is relaxed).

Thus, in accordance with the invention, the processing unit 4 processes the output of a suitable sensor and identifies a time for the pain threshold measurement. The type of sensor and/or processing required depends on how the suitable time is defined (e.g. based on a low heart rate, low level of physical activity, and/or appropriate leg posture), but in some embodiments the processing unit 4 can monitor the subject 10 and identify a suitable time using the measurements from the sensor 8 (particularly where the sensor 8 is an accelerometer and/or a stretch sensor). Alternatively (and particularly in the embodiments where the sensor 8 is an electrode for EMG), the processing unit 4 can obtain the required measurements from a separate sensor that is located at the same or a different position on the body of the subject. In some embodiments, multiple sensors can be provided on the subject for measuring the activity and/or posture of the subject. For example, an accelerometer can be provided on the lower leg or at the ankle (e.g. integrated with the electrode(s) 6) and/or on the upper body of the subject, in addition to the sensor 8 at the thigh of the subject, to improve the position/posture detection.

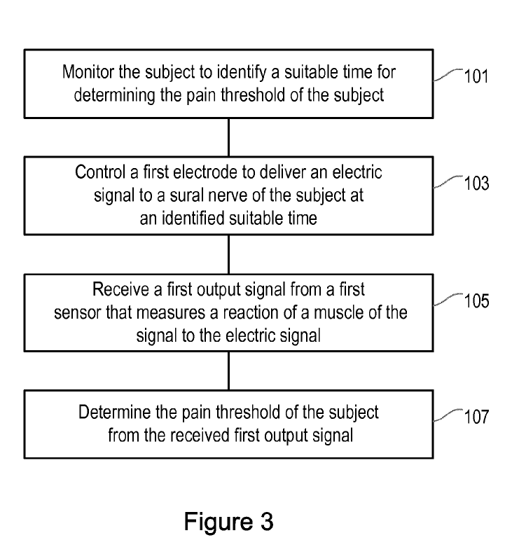

It will be appreciated that FIG. 1 only shows the components required to illustrate this aspect of the invention, and in a practical implementation the apparatus 2 will comprise additional components to those shown. For example, the apparatus 2 may comprise a battery or other power supply for powering the apparatus 2, a communication module for enabling the measurements of the pain threshold of the subject to be communicated to a base unit for the apparatus 2 or a remote computer, and/or one or more user interface components that allow the subject or another user to interact and control the apparatus 2. As an example, the one or more user interface components could comprise a switch, a button or other control means for activating and deactivating the apparatus 2 and/or pain threshold measurement process. The user interface components can also or alternatively comprise a display or other visual indicator for providing information to the subject and/or other user about the operation of the apparatus 2, including displaying the measurements of the pain threshold. The flow chart in FIG. 3 illustrates a method of determining a pain threshold of a subject according to an aspect. In this method one or more electrodes 6 have been placed on or applied to the subject 10 to enable an electric signal to be delivered to the sural nerve, and the sensor 8 is positioned on the subject 10 to enable movements (e.g. contractions, twitches, etc.) of the vastus lateralis to be measured. Each of the steps of the method can be performed by the processing unit 4.

In a first step, the subject 10 is monitored to identify a suitable time for determining the pain threshold of the subject 10 (step 101). In some embodiments, this step comprises processing a signal output by the sensor 8 that is used to measure the movements of the vastus lateralis to identify the suitable time. In other embodiments, this step comprises processing a signal output by a sensor other than sensor 8 to identify the suitable time.

In some embodiments, this step comprises monitoring the heart rate of the subject, and a suitable time for determining the pain threshold of the subject can be determined as a time in which the heart rate of the subject is below a threshold. In some embodiments, an average of the heart rate over a period of time (e.g. a few seconds, or several minutes) can be determined, and this average can be compared to the threshold. The threshold can be set based on the characteristics of the subject (e.g. it could be set taking into account the resting heart rate for the subject). Alternatively the threshold can be set based on population averages. As an alternative, the threshold can take a preset value, e.g. 60 beats per minute, bpm.

In further or alternative embodiments, step 101 can comprise monitoring the movements of a muscle of the subject 10 and a suitable time for determining the pain threshold of the subject can be identified as a time in which the amount of movement or an activity level of the muscle of the subject is below a threshold. In some embodiments, an average of the activity level over a period of time can be determined, and this average compared to the threshold. The muscle monitored in this embodiment can be the vastus lateralis, another muscle in the leg of the subject 10 or another muscle in the body of the subject 10.

In further or alternative embodiments, step 101 can comprise monitoring a posture of the leg of the subject 10 and identifying a suitable time for determining the pain threshold of the subject as a time in which the knee of the subject 10 is bending at a particular angle or within a predetermined range of angles. The angle or range of angles can be pre-set, subject-dependent and/or be determined during a calibration phase of the apparatus 2. This embodiment can also comprise determining the posture of another part of the body of the subject, e.g. the torso, to determine if the subject is reclined or lying down (which is preferred for the pain threshold measurement).

Once a suitable time has been identified, an electric signal is delivered to the sural nerve of the subject 10 during the identified time using the electrode(s) 6 (step 103). After the electric signal is delivered to the sural nerve of the subject 10, an output signal is received from the sensor 8 that is measuring a reaction of a muscle of the subject to the electric signal (step 105).

The output signal is then processed to determine the pain threshold of the subject 10 (step 107). Briefly, the output signal is processed to determine if the muscle twitched as a result of the application of the electric signal, and the level of the electric signal (current) that produced the muscle twitch provides the pain threshold for the subject 10. If no twitch is detected in the output signal, then the method returns to step 103 and increases the stimulation level (i.e. delivers an electric signal with a higher current). The first stimulation level that produces a detectable twitch in the output signal (or a twitch having a magnitude above a threshold value) provides the pain threshold for the subject 10 (i.e. the pain threshold equals the stimulus level).

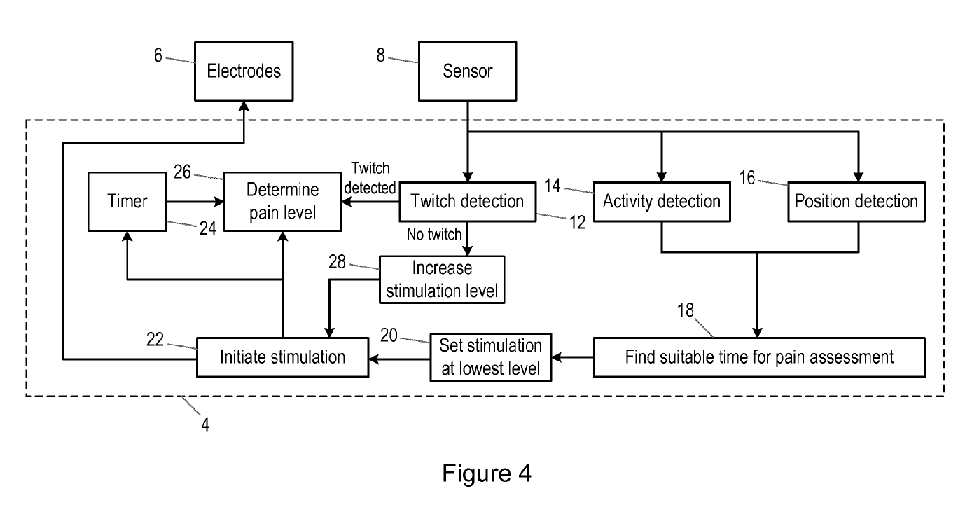

FIG. 4 illustrates various processing stages performed by the processing unit 4 according to an exemplary embodiment. In this figure, the processing unit 4 is connected to the electrode(s) 6 and sensor 8. The signals from the sensor 8 (e.g. acceleration measurements or stretch measurements) are used both for detecting twitches and for detecting a suitable time in which to stimulate the sural nerve. Thus, the signals from the sensor 8 are input to three detection stages or modules, twitch detection module 12, activity detection module 14 and position (and/or posture) detection module 16.

Twitches that occur as a result of stimulation of the sural nerve have a specific pattern that can be distinguished from larger movements such as walking. When a muscle contracts, as is the case during stimulation of the afferent nerve, the muscle moves outward in a short burst. This movement is distinguishable from other movements when found directly following a stimulus, and is different from the relative large, slower, and longer habitual movements such as walking. As a result, twitch detection stage 12 is able to identify twitches in acceleration measurements or stretch measurements.

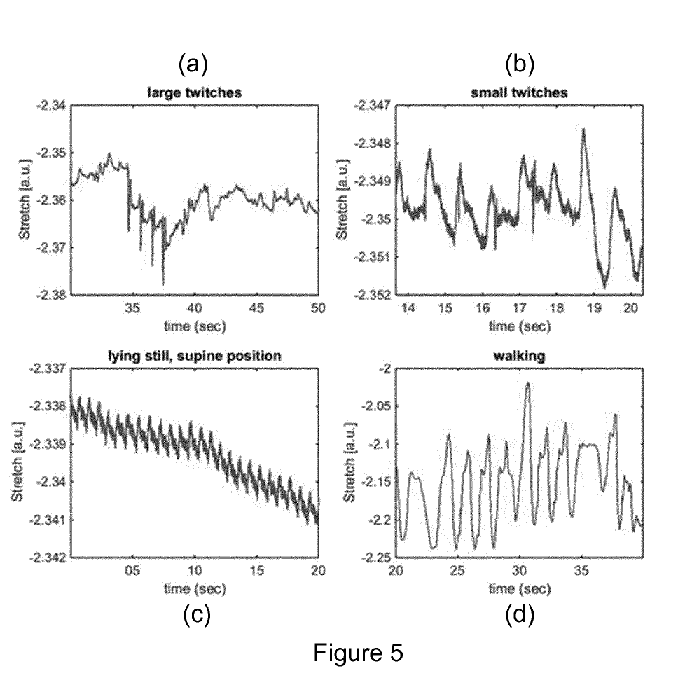

The graphs in FIG. 5 illustrate some exemplary signals obtained using a stretch sensor that is attached to the thigh of a subject. FIG. 5(a) illustrates a measurement signal from a stretch sensor when large twitches are present between 34 and 38 seconds, with the knee of the subject bent at 30 degrees, FIG. 5(b) illustrates a measurement signal from a stretch sensor when three small twitches are present between 15 and 18 seconds, FIG. 5(c) illustrates a measurement signal from a stretch sensor when a subject is lying still in a supine position, and FIG. 5(d) illustrates a measurement signal from a stretch sensor when the subject is walking. The fluctuations other than the twitches in FIGS. 5(a) and (b) have a different waveform and are at least partly caused by pulses of the arteries. These influences of the heart beat can clearly be seen in FIG. 5(c), where the subject is lying still in a supine position. The differences between walking and twitches in terms of waveform and amplitude of the peaks can clearly be seen by a comparison of FIG. 5(d) to FIGS. 5(a) and (b).

The twitches shown in FIGS. 5(a) and (b) were simulated using the patellar reflex or knee-jerk. These graphs clearly show that, on top of other motion and heart beat effects, the twitches can clearly be identified. Thus, twitch detection stage 12 can comprise filtering and/or a peak detector (e.g. a peak shape matching algorithm) that is tuned or configured to the particular shape of a peak caused by a muscle twitch and can easily detect twitches and distinguish them from other information present in the signal. Filtering can comprise high-pass filtering or filtering using the second order derivative of a Gaussian. Those skilled in the art will be aware of other techniques that can be used for identifying twitches in a stretch sensor signal.

In a similar way, there are differences between the waveforms for large twitches, small twitches, different postures and walking when an accelerometer is used as the twitch sensor 8, and thus twitch detection stage 12 can be ‘tuned’ to detect acceleration peaks associated with twitches.

The activity detection module 14 and position (and/or posture) detection module 16 detect the level of activity of the leg (or more generally of the subject 10) and detect the position/posture of the leg respectively and output appropriate signals to processing stage 18.

The activity detection module 14 can detect large movements of the muscles by analyzing the amplitude and period of the sensor signal. In addition, in some embodiments, the activity detection module 14 can additionally or alternatively detect the heart rate of the subject from the sensor signal.

As shown in FIG. 5 (FIG. 5(c) in particular), the signal from a stretch sensor may also contain information on the heart rate of the subject, and thus activity detection stage 14 can analyze the sensor signal to determine the heart rate. The heart rate can be identified using peak detection and/or peak template matching algorithms, or alternatively or additionally by using filtering techniques, for example second-order derivative Gaussian filters. Those skilled in the art will be aware of suitable techniques for extracting a heart rate signal from accelerometer measurements in embodiments where an accelerometer is used as the twitch sensor 8.

The detection of the position and/or posture of the leg or subject by position detection module 16 can readily be detected from an acceleration signal using techniques known to those skilled in the art. In particular, if the orientation of the accelerometer with respect to the leg is known (e.g. the x-axis is aligned with the thigh bone), it is possible to estimate the position of the leg with some accuracy. In addition, it has been found that the signal from a stretch sensor varies according to the angle of the subject's knee (in particular the circumference of the thigh increases as the knee is bent), and thus the position or posture of the leg can be determined by analysis of changes in a stretch sensor signal over time.

Processing stage 18 processes the outputs of detection stages 14 and 16 to identify a suitable time for performing an assessment of the pain threshold of the subject 10. If a suitable time is identified, processing stage 18 outputs an indication that a pain threshold measurement can be made and the stimulation of the sural nerve is initiated. If processing stage 18 has not yet identified a suitable time to make a pain threshold measurement, processing stage 18 can output an appropriate indication (and the sural nerve is not stimulated).

Detection stages 14, 16 can process measurements from sensor 8 continuously or nearly continuously to determine a suitable time for a measurement. Processing stage 18 can identify a suitable time as the first time where any or both of the activity detection stage 14 and position detection stage 16 indicate that a measurement can be made (e.g. as soon as the activity level is below a threshold, and/or as soon as the leg is in the correct posture). Alternatively, processing stage 18 can identify a suitable time to take the measurement once the activity detection stage 14 and/or the position detection stage 16 have indicated that conditions are suitable for a measurement to be made for a certain time period (e.g. the activity level has been below a threshold for a certain amount of time (e.g. a few seconds or minutes), and/or when the leg has been in the correct posture for a certain amount of time (e.g. a few seconds or minutes)).

If a suitable time has been identified, the output from processing stage 18 results in the setting of a stimulation level for the sural nerve (initially the stimulation level is set to a lowest or default value) at stage 20. The lowest or default value for the stimulation level can depend on the size and type of electrodes and their placement. For example, a lowest or default stimulation value can correspond to an electric signal with a current of just a few mA, for example 1 or 2 mA.

Stimulation of the sural nerve is then initiated by stimulation stage 22 which outputs the appropriate electric signal to electrode(s) 6. Stage 22 also indicates to a timer module 24 that the stimulation is being applied, and the timer module 24 measures the time that has elapsed since the electric signal was applied to the sural nerve. Stage 22 also indicates that the stimulation is being applied to a pain threshold measurement module 26.

The patellar reflex latency is about 21 milliseconds. It can therefore be expected that the onset of a detected twitch, say, between 10 to 40 milliseconds after the electric stimulus was applied to the sural nerve can be considered to have resulted from the electric signal. The pain threshold measurement module 26 therefore compares the timing of a twitch detected by twitch detection module 12 to the time since the electric signal was applied (as indicated by timer module 24), and a detected twitch that occurs in a particular time window (e.g. between 10 to 40 milliseconds) can be considered to result from the electric signal. Twitches falling outside this window will be discarded and not used for pain threshold measurement.

If a suitable twitch is detected, pain threshold measurement module 26 analyses the twitch to determine a measure of the pain threshold of the subject 10 (e.g. the pain threshold measurement module 26 determines the pain threshold of the subject 10 as the stimulation level that caused the detected twitch).

As noted above, twitch detection stage 12 analyses the output signal from the sensor 8 to detect if the muscle has twitched as a result of the stimulation of the sural nerve. If no twitch is detected, the stimulation level is increased (at processing stage 28), and stimulation stage 22 applies the increased electric signal to the sural nerve of the subject 10 via the electrode(s) 6. This ‘loop’ continues until a twitch is detected by twitch detection stage 12, which outputs an indication that a twitch has been detected to pain threshold measurement module 26 that analyses the detected twitch to determine the pain threshold of the subject (e.g. the pain threshold measurement module 26 determines the pain threshold of the subject 10 as the stimulation level that caused the detected twitch).

In a similar way to the setting of the lowest or default stimulation level, the amount by which the stimulation level is increased at processing stage 28 can depend on the size and type of electrodes and their placement. For example, the stimulation value can be increased by just a few mA each time, say 1 or 2 mA.

Likewise, if the pain threshold measurement module 26 discards a detected twitch (since it falls outside an acceptable time window), the stimulation level can be increased by processing stage 28 and the increased stimulation applied to the sural nerve.

In embodiments where information on heart rate is available (e.g. through processing of the sensor signal from sensor 8 or from another sensor), the pain threshold measurement module 26 can also make use of the heart rate information when determining the pain threshold since heart rate can be an indicator of pain felt by a subject.

It will be appreciated that while the stimulation increase/twitch detection loop is being performed, processing stage 18 can continue to monitor the output of activity detection stage 14 and position detection stage 16 to determine if the conditions are still suitable for a pain threshold measurement. If processing stage 18 determines that conditions are not suitable (e.g. the activity level is too high), then the processing stage 18 can provide a suitable indication to stimulation module 22 so that stimulation of the sural nerve is stopped. If the processing stage 18 subsequently determines that conditions are again suitable for a measurement of the pain threshold, stimulation of the sural nerve can be resumed, either at the last used stimulation level or at the lowest or default level.

There is therefore provided an improved method and apparatus for determining a pain threshold of a subject.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. An apparatus for determining a pain threshold of a subject, the apparatus comprising:
a processing unit adapted to:
monitor the subject to identify a suitable time for determining the pain threshold of the subject;
control a first electrode to deliver an electric signal to a sural nerve of the subject at an identified suitable time;
receive a first output signal from a first sensor that measures a reaction of a muscle of the subject to the electric signal; and
determine the pain threshold of the subject from the received first output signal;
wherein the processing unit is configured to, based on a received sensor output, monitor at least one of i) a heart rate of the subject, ii) movements of the muscle of the subject, and iii) a posture of the leg of the subject, in order to identify the suitable time for determining the pain threshold of the subject.

2. An apparatus as claimed in claim 1, wherein the first sensor is an accelerometer, a stretch sensor, a strain-gauge sensor or a second electrode.

3. An apparatus as claimed in claim 1, wherein the sensor output is an output signal from the first sensor.

4. An apparatus as claimed in claim 1, wherein the processing unit is adapted to receive the sensor output from a second sensor.

5. An apparatus as claimed in claim 4, wherein the second sensor is an accelerometer.

6. An apparatus as claimed in claim 1 wherein the processing unit is further adapted to compare the heart rate of the subject to a threshold, and to identify the suitable time for determining the pain threshold of the subject as a time at which the heart rate of the subject is below the threshold

7. An apparatus as claimed in claim 1 wherein the processing unit is further adapted to identify the suitable time for determining the pain threshold of the subject as a time in which an activity level of the muscle of the subject is below a threshold.

8. An apparatus as claimed in claim 1 wherein the processing unit is further adapted to identify the suitable time for determining the pain threshold of the subject as the time in which the knee of the subject is bending at an angle that is within a predetermined range of angles.

9. An apparatus as claimed in claim 1, wherein the apparatus further comprises the first electrode and/or the first sensor.

10. A method of determining a pain threshold of a subject, the method comprising:
monitoring the subject to identify a suitable time for determining the pain threshold of the subject;
controlling a first electrode to deliver an electric signal to a sural nerve of the subject at an identified suitable time;
receiving a first output signal from a first sensor that measures a reaction of a muscle of the subject to the electric signal; and
determining the pain threshold of the subject from the received first output signal;
wherein monitoring the subject to identify the suitable time for determining the pain threshold of the subject comprises monitoring at least one of i) a heart rate of the subject, ii) movements of the muscle of the subject, and iii) a posture of the leg of the subject.

11. A method as claimed in claim 10, wherein the first sensor is an accelerometer, a stretch sensor, a strain-gauge sensor or a second electrode.

12. A method as claimed in claim 10, wherein the step of monitoring the subject to identify a suitable time for determining the pain threshold of the subject comprises processing an output signal from the first sensor to identify the suitable time.

13. A method as claimed in claim 10, wherein the method further comprises the step of:
receiving a second output signal from a second sensor; and
wherein the step of monitoring the subject to identify a suitable time for determining the pain threshold of the subject comprises processing the second output signal to identify the suitable time.

14. A method as claimed in claim 13, wherein the second sensor is an accelerometer.

15. A computer program product comprising a computer readable medium having computer readable code embodied therein, the computer readable code being configured such that, on execution by a suitable computer or processor, the computer or processor is caused to perform the method of claim 10.
